(19) United States
(12) Patent Application Publication
(10) Pub. No.: US 2020/0178954 A1
(43) Pub. Date: Jun. 11, 2020

(54) FLAT SURGICAL SUTURE CONSTRUCTS WITH LOOPS AND METHODS OF TISSUE FIXATION

(71) Applicant: Arthrex, Inc., Naples, FL (US)

(72) Inventor: Stephen S. BURKHART, Beorne, TX (US)

(21) Appl. No.: 16/788,160

(22) Filed: Feb. 11, 2020

Related U.S. Application Data
(63) Continuation of application No. 16/011,059, filed on Jun. 18, 2018.

(51) Int. Cl. A61B 17/04

(52) U.S. Cl. CPC A61B 17/0401; A61F 2/0811

(57) ABSTRACT

Knotless surgical constructs and methods of tissue repairs. A surgical construct can offer both repair and shuttling capabilities to allow for a single pass to load multiple sutures at once. A surgical construct is in the form of a flat suture tape provided with reinforced perforations and/or shuttling loops that can pass or shuttle multiple repair sutures while making a single pass through or around soft tissue.

### CROSS-REFERENCE TO RELATED APPLICATIONS

This application is a continuation of, and claims priority benefit from, U.S. application Ser. No. 16/011,059, filed Jun. 18, 2018, the disclosure of which is incorporated by reference in its entirety herein.

### BACKGROUND

The disclosure relates to surgical devices and, more specifically, to flat surgical suture constructs and associated methods of tissue repairs.

### SUMMARY

Surgical constructs with shuttling loops and/or perforations and methods of tissue repairs are disclosed. A surgical construct can offer both repair and shuttling capabilities to allow for a single pass to load multiple flexible strands at once. The surgical construct can pass or shuttle multiple repair sutures while making a single pass through or around soft tissue. The surgical construct is provided with a plurality of shuttling perforation that are reinforced to allow additional flexible strands to pass through the shuttling perforations to compress adjacent tissue at any angle. The surgical construct also allows for collagen ingrowth through the shuttling perforations. The surgical construct may be knotless.

Methods of tissue repair are also disclosed. In an embodiment, a flat suture construct is provided with multiple reinforced perforations or loops to allow multiple flexible strands such as sutures or tapes to be shuttled and passed simultaneously without individually passing the flexible strand through tissue more than one time.

### BRIEF DESCRIPTION OF THE DRAWINGS

FIG. 1 illustrates a surgical construct according to an exemplary embodiment.

FIG. 2 illustrates a surgical construct according to another exemplary embodiment.

FIG. 3 illustrates a surgical construct according to another exemplary embodiment.

FIG. 4 illustrates a surgical construct according to another exemplary embodiment.

FIG. 5 illustrates a surgical construct according to another exemplary embodiment.

### DETAILED DESCRIPTION

The disclosure provides surgical constructs for passing or shuttling at least one length of flexible material, for example suture such as high strength suture, tape, wire, cable, or fabric. The surgical constructs include a flat suture such as a flat suture tape provided with at least one shuttling loop that allows additional flexible strands to pass through the construct and through tissue. The surgical construct may be knotless. The surgical construct may be part of a knotless construct and/or may be employed in conjunction with a knotless construct.

The surgical constructs permit attachment of multiple sutures through the tape. The ability to pass additional sutures through the tape would permit multiple suture strands and FiberTape® strands to be loaded at a tissue repair site without the requirement of individually passing the suture through the tissue more than once (more than one time). Surgical repairs with improved suture tape such as improved FiberTape® of the present disclosure (and as detailed below) provide additional compression area and angles of compression at a repair site.

The new suture tape configuration is a flat suture tape that has a series of features that facilitate unique features to be incorporated into the body of a flat suture tape itself, and these unique tape constructs provide for improved methods of tissue repair and multiple suture strand loading techniques with a single suture needle passage. In addition, the perforations allow collagen to bridge more easily between adjacent layers of tissue that are compressed separately by such perforated tapes. For example, when a Superior Capsular Reconstruction with dermal allograft is secured laterally in the humerus with perforated tapes and is in direct contact with an overlying layer of rotator cuff tissue, there is a greater surface area of contact between the graft and the rotator cuff tissue (due to the additional contact area through the perforations), thereby enhancing the chance of healing between the two layers.

The novel flat suture constructs may be exemplary suture tapes of various configurations that allow multiple suture strands/tapes to pass through the flat suture constructs. The flat suture constructs are provided with perforations and/or loops extending from the flat suture. The flat suture may be braided flat suture such as FiberTape® suture tape.

In one embodiment, the tape is provided with multiple “shuttling perforations” or loops or eyelets that are reinforced by stitching (as for a button hole). The reinforcement allows for additional sutures or tapes to pass through the perforations, to compress adjacent tissue at any angle and to allow for collagen ingrowth through the perforations.

In another embodiment, “appendage loops” are provided to shuttle tapes or sutures for bridging or for angled compression of soft tissues.

In yet additional embodiments, sequential shuttling loops are provided alone or in combination with the reinforced perforations in tape. The sequential loops may extend from both terminal ends of the flat braided tape. The loops may be employed as shuttling loops, through which additional sutures may be passed to create improved tissue repair constructs.

The perforations provide a site where additional suture or strands or tapes may be passed/threaded through the flat suture. The perforations may be used in a variety of ways to improve fixation of a desired construct at a repair site or to accommodate any tissue repair angle. The reinforced perforations may be also advantageously used by a surgeon to attach a clamp or other surgical tool to more easily manipulate or pull the suture into a desired position.

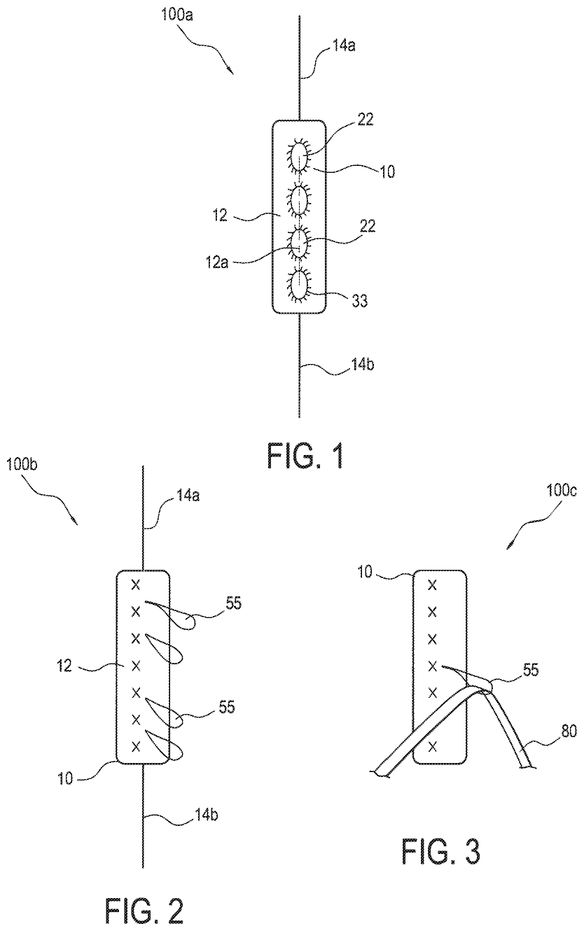

Referring now to the drawings, where like elements are designated by like reference numerals, FIGS. 1-5 illustrate structural components of surgical construct 100a, 100b, 100c, 100d, 100e (flat suture 100a, 100b, 100c, 100d, 100e; flat suture construct 100a, 100b, 100c, 100d, 100e; tape construct 100a, 100b, 100c, 100d, 100e) of the present disclosure.

Flat suture constructs 100a, 100b, 100c, 100d, 100e may be products in the form of tapes such as flat suture tapes that may be fabricated from a sheet of material or from strips of suture material. The flat suture tape constructs detailed below may be non-braided tape sutures or braided tape sutures.

FIG. 1 illustrates surgical construct 100a in the form of a flat braided tape-like suture 10 (suturing construct 10 or flat tape 10) that includes a perforation 22, or series of perforations 22. Perforations 22 may be any holes, openings, eyelets, closed loops, shuttling loops, orifices, ports or any similar structures (and combinations thereto) that allow one or more strands to pass therethrough.

Perforations 22 may be further reinforced around the perimeter of the perforation by threading around the opening, much like that of a button hole of a garment, to form reinforced perforations 33. Perforations 33 provided along the length of the flat suture tape 10 permit for the shuttling of other sutures (flexible strands or similar elements such as suture tapes, suture passing constructs, etc.) through the tape so as to provide “shuttling” perforations. The perforations may also be used to attach additional strands of suture to permit additional compression of a suture construct created at a repair site, such as a rotator cuff repair site. Additional compression of tissue at any angle would therefore be permitted by virtue of the additional threaded strands through the perforations. The perforations in the suture tape also facilitate improved healing at the repair site, permitting collagen ingrowth through the perforations (or other growth/repair material through the perforations).

The multiple reinforced perforations 33 in tape are reinforced by stitching, as for a button-hole. This aspect allows for additional sutures or tapes to pass through the perforations in order to compress adjacent tissue at any angle while also allowing for collagen ingrowth through the perforations.

As illustrated in FIG. 1, suturing construct 10 includes a middle region 12 with two tail regions 14a, 14b; and four exemplary-only reinforced perforations/shuttling loops 33 (eyelets 33 or closed loops 33), each loop 33 being provided extending through the middle region 12 and along a longitudinal axis 12a of the middle region 12. One or more flexible strands may pass through at least one of the two shuttling loops 33. The middle region 12 may be a flat suture section. The middle region may have a gradual taper. The two tail regions 14a, 14b may be formed of round suture, with same or different diameter. In an exemplary embodiment, perforations 33 (shuttling loops 33) are located along longitudinal axis 12a of middle region 12 and about symmetrically located relative to the longitudinal axis 12a. In an exemplary embodiment, perforations 33 (shuttling loops 33) are located along longitudinal axis 12a of middle region 12 and spaced apart from each other by a distance “d”. In an exemplary embodiment, perforations 33 (shuttling loops 33) are not located along longitudinal axis 12a but about symmetrically relative to the longitudinal axis 12a.

The tail regions 14a, 14b may have similar or different diameters. As illustrated in FIG. 1, each of the diameters of the tail regions 14a, 14b is smaller than the width of the middle region 12. Tail regions 14a, 14b may have similar or different lengths. One or both of the tail regions 14a, 14b may have a very fine end that is coated, impregnated, or stiffened with a material such as plastic, for example. One or both of the tail regions 14a, 14b may include a tapered end that terminated in a very fine end.

FIG. 1 also illustrates four shuttling loops 22 that are all reinforced to form four reinforced shuttling loops 33. In the exemplary embodiment of FIG. 1, loops 33 are provided symmetrically relative to the middle region 12 and extending along midline 12a. Shuttling loops 22, 33 are perforations that extend from one surface of the middle region 12 to another surface of the middle region 12, for example, from an upper surface to a lower surface of the middle region 12. Shuttling loops 22, 33 may have similar or different diameters and/or similar or different perimeters. As shown in FIG. 1, all shuttling loops 22 are reinforced; however, the disclosure contemplates embodiments wherein any number of shuttling loops is reinforced. The suturing construct may also include non-reinforced shuttling loops adjacent to reinforced shuttling loops. The number of non-reinforced shuttling loops may be similar or different from the number of reinforced shuttling loops.

Shuttling loops 33 allow at least another flexible strand (not shown in FIG. 1) to pass therethrough. Flexible strand may be any additional repair strand, for example, suture strands, suture tapes, nitinol strands, FiberLink™ among many others. One or more flexible strands may pass through one or more shuttling loops 22, 33.

Middle region 12 may have cross-sections of various forms and geometries, including rectangular or flat, among others, or combination of such forms and geometries. In an exemplary embodiment only, middle region 12 may be provided as a braided suture tape, or as a combination of tape and round suture for its tails. The diameter of middle region 12 may be constant or may vary. The middle region 12 can be any suture strand or suture tape, for example, Arthrex FiberTape®, which is a high strength suture tape that is braided and rectangular-like in cross section and as disclosed in U.S. Pat. No. 7,892,256, the disclosure of which is incorporated by reference in its entirety herein. However, the surgical constructs 100a, 100b, 100c, 100d, 100e can be used with any type of flexible material or suture known in the art.

FIGS. 2 and 3 detail surgical construct 100b, 100c in the form of a flat braided tape-like suture 10 with middle region 12 that includes along the length of the midline 12a of the suture an interwoven loop 55 extending therefrom, or series of loops 55 extending therefrom. These loops are described as “appendage” loops 55. The loops are different from other loops provided with available FiberTape® products. The loop provided with a FiberTape® product is located at the terminal end of the tape, and is not provided along the midline of the tape body. FIG. 3 shows a tape 80 (another flexible strand 80) looped through an appendage loop 55.

Interwoven loops 55 (closed loops 55 or eyelets 55) of FIG. 2 are provided attached the middle region 12 and located symmetrically relative to the shuttling loops 22. Interwoven loops 55 may be also asymmetric. Interwoven loops 55 are flexible, continuous loops that may have similar or different diameters and/or similar or different perimeters. Loops 55 may be formed of suture or any similar material. In an embodiment, loops 55 are formed of same material that forms tail regions 14a, 14b, for example, round suture. Loops 55 may be formed of a high strength suture material with surgically-useful qualities such as Arthrex FiberWire® suture disclosed in U.S. Pat. No. 6,716,234, the entire disclosure of which is incorporated herein by reference. In an embodiment, at least one of loops 55 is flexible. In an embodiment, at least one of four exemplary loops 55 is formed of a material (for example, alloy or metal) which does not itself has to be flexible but allows the suturing construct to “flex.”

Loops 55 also allow at least another flexible strand to pass therethrough. As in the previous embodiment, the flexible strand may be any additional repair strand, for example, suture strands, tapes, nitinol strands, FiberLink™ among many others. One or more flexible strands may pass through one or more shuttling loops 22 and/or through one or more loops 55.

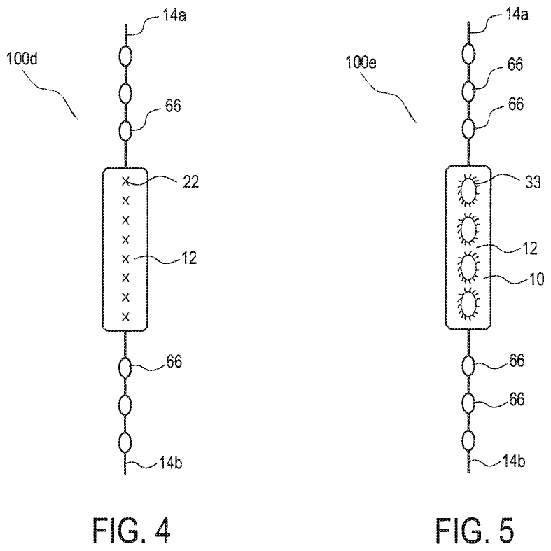

FIG. 4 illustrate surgical construct 100d which is similar in part to constructs 100a, 100b, 100c detailed above, but differs in that construct 100d is in the form of a flat braided tape-like suture 10 having a series of sequential shuttling loops 66 extending from both terminal ends of the flat braided tape 10. In an exemplary embodiment, sequential shuttling loops 66 are provided along at least one of tails 14a, 14b, preferably along both tails 14a, 14b. Each of the loops 66 may be employed as shuttling loops, as additional suture/flexible materials may be passed in creating improved tissue reconstructive and repair constructs.

FIG. 5 provides surgical construct 100e in the form of a flat braided tape-like suture 10 having a series of perforations 33 along the body of the suture 10, as well as a series of sequential shuttling loops 66 extending from both terminal ends of the flat braided tape 10. The perforations 33 may also be reinforced around the perimeter, much like a button hole of a garment. The perforations may be all or none reinforced, or a combination of reinforced and non-reinforced. The perforations provide a site where additional suture or a strand of FiberTape® may be threaded through the flat suture, and used in any variety of ways to improve fixation of a desired construct at a repair site or to accommodate virtually any obscure tissue repair angle. The reinforced perforations may also be advantageously used by the surgeon to attach a clamp or other surgical tool to more easily manipulate or pull the suture into a desired position.

In an embodiment, the surgical construct is provided with one or more shuttling loops. In an embodiment, at least one additional flexible strand is passed simultaneously with the surgical construct. In an embodiment, the surgical construct is a flat suture tape with one or more shuttling loops or perforations. In an embodiment, the surgical construct is a flat suture tape with one or more shuttling loops or perforations, and at least a flexible strand or material passed through at least one of the two or more shuttling loops or perforations. In an embodiment, the perforations are reinforced.

In an embodiment, one or more additional flexible strands are passed through one or more shuttling loops. In an embodiment, the surgical construct is pre-loaded on a fixation device such as a knotless fixation device to be secured within hard tissue such as bone. In an embodiment, the fixation device has a closed eyelet and the surgical construct is passed through the eyelet. In an embodiment, the fixation device has a closed eyelet and the surgical construct with at least another additional flexible strand are passed through the eyelet.

In an embodiment, a surgical construct comprises: a suturing construct with a middle region and two ends; one or more shuttling loops; and one or more appendage loops. The surgical construct may be knotless. The middle region may be a flat suture section. The suture section may be suture tape which may have a gradual tape. The two ends may be opposed ends. The shuttling loops may be provided symmetrically or asymmetrically relative to a longitudinal axis of the middle region. The shuttling loops may provide passing of one or more lengths of flexible material, for example, strands such as suture, for example, high strength suture, tape, suture tape, combination of suture and tape, wire, cable, or fabric, among many others. One or more lengths of flexible material may pass through one or more shuttling loops.

The appendage loops may be provided symmetrically or asymmetrically relative to a longitudinal axis of the middle region. The appendage loops may provide passing of one or more lengths of flexible material, for example, strands such as suture, for example, high strength suture, tape, suture tape, combination of suture and tape, wire, cable, or fabric, among many others. One or more lengths of flexible material may pass through one or more appendage loops.

In another embodiment, a knotless surgical construct comprises: a flexible construct with a middle region and two tail regions; two or more shuttling loops, each loop being provided through/on the middle region; and sequential shuttling loops located along one or two of the tail regions. At least another flexible strand may be passed through at least one of the two shuttling loops and/or the sequential shuttling loops. The flexible construct may be a repair suture. The middle region may be a suture tape. The flexible strand may be suture such as high strength suture, tape, suture tape, combination of suture and tape, wire, cable, or fabric, among many others. The tail regions may be round suture.

Methods of tissue repair are also disclosed. In an embodiment, additional flexible strands (additional repair strands) can be passed simultaneously with the flat suture construct by conducting a single pass through tissue to be repaired (ligament, tendon, graft, etc.). The flat suture construct (repair suture) and the additional flexible strands may be passed and/or shuttled simultaneously with a surgical instrument such as a suture passer. The flat suture construct and the additional flexible strands may be passed or shuttled simultaneously without loading a separate shuttling stitch or wire, and without passing each strand independently. The flat suture construct may include a middle region with two ends and two or more shuttling loops and/or reinforced perforations and/or sequential shuttling loops located on the tail regions. The middle region may be in the form of a flat suture tape. The shuttling loops and/or reinforced perforations and/or sequential shuttling loops facilitate shuttling the additional flexible strands (additional repair sutures) simultaneously with the flat suture construct. One or more additional flexible strands may pass through one or more shuttling loops and/or one or more reinforced perforations and/or one or more sequential shuttling loops.

A method of tissue repair (for example, soft tissue to bone fixation) includes passing a surgical construct through tissue and securing the tissue with the surgical construct. The surgical construct may be passed through or around tissue by loading the surgical construct onto a suture passer and simultaneously shuttling a repair strand together with additional repair strands. The additional repair strands are passed through shuttling loops of the surgical construct and passed/shuttled through or around tissue without the need to pass each additional repair strand individually. A single pass loads multiple repair strands at once.

The surgical constructs 100a, 100b, 100c, 100d, 100e may be employed for various tissue repairs, for example, soft tissue to bone repairs, or soft tissue to soft tissue repairs, among many others. The tissue repairs may employ at least one knotless fixation device. The knotless fixation devices may be knotless anchors, for example, swivel and/or screw-in suture anchors and/or push-in anchors (such as an Arthrex SwiveLock® anchor or a PushLock® Anchor). In an exemplary embodiment only, the fixation device is a knotless suture anchor such as the two-piece Arthrex PushLock® anchor, disclosed in U.S. Pat. No. 7,329,272, or an Arthrex SwiveLock® anchor, disclosed in U.S. Pat. No. 8,012,174 issued Sep. 6, 2011, U.S. Pat. No. 9,005,246 issued Apr. 14, 2015, and US 2013/0296936 published Nov. 7, 2013, the disclosures of all of which are fully incorporated by reference in their entirety herein.

According to an exemplary embodiment only, the surgical constructs 100a, 100b, 100c, 100d, 100e of the present disclosure may be also employed with knotted fixation devices, for example, knotted anchors. Thus, the disclosure is not limited to the use of suturing constructs 100a, 100b, 100c, 100d, 100e with knotless fixation devices such as two-piece Arthrex PushLock® anchor, and the disclosure contemplates the use of constructs 100a, 100b, 100c, 100d, 100e with any type of fixation device, knotless or knotted or combination of knotless and knotted fixation devices. The exemplary constructs of the present disclosure may be employed in various tissue repairs such as knotless rotator cuff repair with the SpeedBridge™ and SpeedFix™ repair techniques, or similar reattachment techniques of soft tissue to bone employing knotless fixation devices for the formation of single, double or multiple row constructs in arthroscopic rotator cuff repairs, or high demand applications like AC joint reconstruction and other areas where tissue pull-through may be a concern.

Strands (such as strand 80) that may be passed through perforations 22, 33, 55, 66 may be made of any known suture construct, such as multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby, incorporated by reference in its entirety herein). FiberWire® suture is formed of an advanced, high-strength fiber material, namely ultrahigh molecular weight polyethylene (UHMWPE), sold under the tradenames Spectra (Honeywell) and Dyneema (DSM), braided with at least one other fiber, natural or synthetic, to form lengths of suture material. The preferred FiberWire® suture includes a core within a hollow braided construct, the core being a twisted yarn of UHMWPE. The strands may be also formed of suture tape, for example, Arthrex FiberTape®, which is a high strength suture tape that is braided and rectangular-like in cross section and as disclosed in U.S. Pat. No. 7,892,256, the disclosure of Which is incorporated by reference in its entirety herein, Surgical constructs 100a, 100b, 100c, 100d, 100e can be used with any type of flexible material or suture known in the art.

According to an exemplary embodiment only, one or more surgical constructs 100a, 100b, 100c, 100d, 100e of the present disclosure may be employed in a method for double row fixation of tendon to bone, as detailed in U.S. Pat. No. 8,012,174, the disclosure of which is fully incorporated by reference in its entirety herewith. A method of exemplary tissue fixation with the surgical construct 100 comprises inter alia the steps of: (i) providing surgical construct 100a, 100b, 100c, 100d, 100e pre-loaded (manufactured) on a fixation device (for example, a knotless fixation device); (ii) securing the fixation device with the pre-loaded construct 100a, 100b, 100c, 100d, 100e into a bone socket or tunnel; (iii) passing the surgical construct 100a, 100b, 100c, 100d, 100e through tissue to be repaired; and (iv) employing an additional strand 80 (passed through one or more perforations/shuttling loops 22, 33, 55, 66) to fixate tissue.

The surgical constructs 100a, 100b, 100c, 100d, 100e described above may be formed of strands of a high strength suture material with surgically-useful qualities, including knot tie down characteristics and handling, such as Arthrex FiberWire® suture disclosed in U.S. Pat. No. 6,716,234, the entire disclosure of which is incorporated herein by reference. The surgical suture tape constructs may be formed of optional colored strands (preferably black) to assist surgeons in distinguishing between suture lengths with the trace and suture lengths without the trace. Preferably, each of tail regions 14a, 14b may be provided in different colors to assist surgeons in retrieving one tail from each of the knotless fixation devices and then loading them through another fixation device, during the formation of a criss-cross suturing pattern.

Surgical constructs 100a, 100b, 100c, 100d, 100e may be preferably coated (partially or totally) with wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon, Hostaflon, or others), PBA (polybutylate acid), ethyl cellulose (Filodel) or other coatings, to improve lubricity of the suture or tape, knot security, pliability, handleability or abrasion resistance, for example.

In additional embodiments, middle region 12 may be formed of polyester (for example, braided polyester) and the suture tail 14a, 14b with shuttling loops 22, 33, 55, 66 may be also formed of polyester or a similar material. In an exemplary embodiment only, middle region 12 may be formed of braided polyester as a flat suture tape. In addition, a coating may be provided to the yarns forming the surgical constructs 100a, 100b, 100c, 100d, 100e before braiding. Polyester yarns for the braided construct of the middle region 12 may be coated using a silicone elastomer (or a similar material as detailed above) prior to braiding. Similarly, the suture tail regions 14a, 14b and/or shuttling loops 22, 33, 55, 66 may be coated using the same or different coating material. If desired, at least one of the tail regions 14a, 14b and/or at least one of shuffling loops 22, 33, 55, 66 and/or at least one of the additional flexible strands 80 of construct 100a, 100b, 100c, 100d, 100e (preferably both tail regions 14a, 14b, shuttling loops 22, 33, 55, 66 and additional flexible strands 80) may be coated, impregnated, or otherwise stiffened with a material such as plastic, for example.

The tail regions 14a, 14b, the shuttling loops 22, 33, 55, 66 and the additional flexible strands 80 of the surgical constructs 100a, 100b, 100c, 100d, 100e may be also provided with tinted tracing strands, or otherwise contrast visually with the middle region of the suture construct, which remains a plain, solid color, or displays a different tracing pattern, for example. Accordingly, when the surgical construct is loaded through the eyelet of a suture anchor or passed through tissue, for example, at least one of the tail regions 14a, 14b of the suture construct, or at least one of the shuttling loops 22, 33, 55, 66, or middle region 12, or the additional flexible strand 80, may be visually coded, making identification and handling of the suture legs simpler. Easy identification of suture in situ is advantageous in surgical procedures, particularly during arthroscopic surgeries, such as endoscopy and laparoscopy.

The surgical construct 100a, 100b, 100c, 100d, 100e of the present disclosure has applicability to suture applications that may be employed in surgical procedures such as rotator cuff repair, Achilles tendon repair, patellar tendon repair, ACL/PCL reconstruction, hip and shoulder reconstruction procedures, and applications for suture used in or with suture anchors.

An exemplary method of tissue repair may comprise inter alia the steps of (i) threading at least one additional repair strand 80 through at least one shuttling loop 22, 33, 55, 66 of a surgical construct 100a, 100b, 100c, 100d, 100e; and (ii) passing the at least one additional repair strand 80 and the surgical construct 100a, 100b, 100c, 100d, 100e through or around tissue to be repaired. The at least one additional repair strand 80 may be passed simultaneously with the flat suture tape 12 of the surgical construct 100a, 100b, 100c, 100d, 100e.

The flat suture constructs of the present disclosure detailed above may consist essentially of suture tapes of various configurations that allow multiple suture strands/tapes to pass through the flat suture constructs. The flat suture constructs are provided with perforations and/or loops extending from the flat suture. The flat suture may be braided flat suture such as FiberTape® suture tape. As detailed above, a tape is provided with multiple “shuttling perforations,” i.e., perforations that are reinforced by stitching (as for a button hole). The reinforcement allows for additional sutures or tapes to pass through the perforations, to compress adjacent tissue at any angle and to allow for collagen ingrowth through the perforations. In another embodiment, “appendage loops” are provided to shuttle tapes or sutures for bridging or for angled compression of soft tissues. In yet additional embodiments, sequential shuttling loops are provided alone (similar to FiberChain®) or in combination with the reinforced perforations in tape. The sequential loops may extend from both terminal ends of the flat braided tape. The loops may be employed as shuttling loops, through which additional sutures may be passed to create improved tissue repair constructs. Arthrex FiberChain® is a single stranded, #2 FiberWire® suture strand that transitions to chain links of interwoven FiberWire®. The constructs of the present disclosure provided with sequential shuttling loops (similar to FiberChain®) in the form of a FiberChain® suture chain may be employed, for example, with the forked tip of a SwiveLock® suture anchor for knotless rotator cuff repair.

The perforations provide a site where additional suture or strands or tapes may be passed/threaded through the flat suture. The perforations may be used in a variety of ways to improve fixation of a desired construct at a repair site or to accommodate any tissue repair angle. The reinforced perforations may be also advantageously used by a surgeon to attach a clamp or other surgical tool to more easily manipulate or pull the suture into a desired position.

The perforations may be reinforced around the perimeter with stitching, much like a button hole stitching pattern. Sutures or additional tapes may then be shuttled through any one or all of the perforations in the flat suture tape. The perforations are suitable for accommodating the passage of sutures or additional tapes, such as a FiberTape®.

The surgical suture construct may consist of a flat suture tape that includes one or a series of perforations (“shuttling” perforations) in a rotator cuff repair. The perforations permits addition shuttling tapes, such as FiberTape®, to be threaded through the flat tape perforations of the criss-crossed construct. This provides additional compression of the rotator cuff, as well as greater versatility of angle compression.

The surgical suture construct may consist of a flat suture tape that includes one or a series of loops (“appendage loops”) extending along the length of the tape. The appendage loops may be woven into the tape as part of the flat suture tape. The loops may then be used to thread an additional shuttling tape (such as a FiberTape®) or suture for bridging or to provide for improved compression area coverage and/or angle of compression for soft tissue. The surgical suture construct may be a flat suture tape that includes a loop (appendage loop), through which a FiberTape® is looped/threaded. The loop of the FiberTape® may create a cinch-loop through an appendage loop of a flat suture tape having an appendage loop. The flat suture tape includes one or a series of perforations suitable for accommodating the passage of sutures or additional tapes, such as a FiberTape®. The perforations may be reinforced around their periphery, such as is akin at a button hole.

The flat suture tape may include a series of more than one interconnected loops at each of the proximal ends of the tape. The flat suture tape may include a series of more than one interconnected loops at each of the proximal ends of the tape.

Methods of tissue repair employing the surgical suture construct that includes a repair suture having at least one region comprising a flat suture tape having one or a series of perforations (“shuttling” perforations), through which additional sutures and/or repair strands may be shuttled simultaneously without loading a separate shuttling stitch or wire, and without passing each strand independently. The shuttling perforations may include a reinforced perimeter, akin to a button hole. Alternatively, or in addition to the shuttling perforations, the repair suture may include a flat suture tape having one or more loops (“appendage” loops), that are woven into as part of the repair suture. A repair suture having a perforation and/or perforations as described provides superior increased compression and angle of compression of the construct, and provides for the incorporation of additional tapes, such as FiberTape®, through the shuttling perforations and/or appendage loops.

For the purposes of the present invention, the term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body. In a preferred embodiment of the invention, the suture comprises a high strength suture sold by Arthrex, Inc. under the tradename FiberWire®. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material.

What is claimed is:

1. A suture construct comprising:
a middle region comprising a flat suture tape;
two tail regions fixedly attached to and extending away from opposite ends of the middle region, wherein the tail regions are structured differently from the middle region; and
at least one appendage loop attached to and extending away from the middle region.

2. The suture construct of claim 1, wherein the at least one appendage loop comprises a plurality of appendage loops arranged along a longitudinal axis of the suture tape.

3. The suture construct of claim 2, further comprising at least one perforation that defines a shuttling loop formed through the middle region.

4. The suture construct of claim 3, wherein the at least one perforation comprises a plurality of perforations.

5. The suture construct of claim 4, wherein the plurality of appendage loops are arranged symmetrically with the plurality of perforations on the middle region.

6. The suture construct of claim 1, further comprising at least one perforation that defines a shuttling loop formed through the middle region.

7. The suture construct of claim 6, wherein the at least one perforation is reinforced.

8. The suture construct of claim 1, further comprising at least one flexible strand configured to pass through the at least one appendage loop.

9. The suture construct of claim 8, wherein the at least one flexible strand comprises a suture tape.

10. The suture construct of claim 8, wherein the at least one flexible strand comprises a plurality of flexible strands.

11. The suture construct of claim 1, wherein at least one of the tail regions comprises a plurality of sequential loops formed thereon.

12. The suture construct of claim 1, wherein the middle region is tapered in a direction towards the tail regions.

13. The suture construct of claim 1, wherein the tail regions comprise round sutures.

14. The suture construct of claim 1, wherein at least one of the middle region, the two tail regions, or the at least one appendage loop is visually coded.

15. The suture construct of claim 1, wherein at least part of the flat suture tape is braided.

16. The suture construct of claim 1, wherein the flat suture tape has a rectangular configuration.

17. The suture construct of claim 1, wherein at least part of the flat suture tape has a rectangular cross-section.

18. The suture construct of claim 1, wherein the suture construct is configured to be applied for knotless fixation of tissue.
